(19) United States
(12) Patent Application Publication
(10) Pub. No.: US 2022/0304943 A1
(43) Pub. Date: Sep. 29, 2022

(54) FLOWABLE CANNABINOID COMPOSITIONS HAVING HIGH EFFECTIVE CONCENTRATIONS

(71) Applicant: Canopy Growth Corporation, Smiths Falls, Ontario (CA)

(72) Inventors: Kurt LEVY, Smith Falls (CA); Jonathan MARTIN, Smith Falls (CA); Kaitlin SMITS, Smith Falls (CA); Brian REID, Smith Falls (CA)

(21) Appl. No.: 17/251,015

(22) PCT Filed: Oct. 28, 2020
(86) PCT No.: PCT/CA2020/051445

§ 371 Date: Dec. 10, 2020

Related U.S. Application Data
(60) Provisional application No. 62/926,818, filed on Oct. 28, 2019.

(51) Int. Cl. A61K 31/05; A61K 31/60; A61K 9/00; A24B 15/167

(52) U.S. Cl. CPC A61K 31/05; A61K 31/60; A61K 9/007; A24B 15/167

(57) ABSTRACT

Disclosed herein is a high-effective-concentration cannabinoid-based vape oil that is flowable within a vape device. The high-effective-concentration cannabinoid-based vape oil comprises a cannabinoid-based incipient and a cannabinoid-based target component. The cannabinoid-based incipient has an in-situ concentration that is less than the saturation concentration of the cannabinoid-based incipient. The cannabinoid-based target component has an in-situ concentration that is less than the saturation concentration of the cannabinoid-based target component. The in-situ concentration of the cannabinoid-based incipient and the in-situ concentration of the cannabinoid-based target component, taken together, is greater than the saturation concentration of the cannabinoid-based target component. The cannabinoid-based target component has an in-actio concentration that is greater than the saturation concentration of the cannabinoid-based target component. As such, the cannabinoid-based vape oil has a high-effective concentration of the cannabinoid-based target component.

### CROSS-REFERENCE TO RELATED APPLICATIONS

This application claims priority to and benefit of U.S. Provisional Patent Application Ser. No. 63/046,536 filed on Jun. 30, 2020; and U.S. Provisional Patent Application Ser. No. 62/926,818 filed on Oct. 28, 2019, each of which is hereby incorporated by reference in its entirety.

### TECHNICAL FIELD

The present disclosure generally relates to cannabinoid compositions that are flowable (e.g. in a vape device) and that have high effective concentrations (e.g. when inhaled by a user).

### BACKGROUND

Cannabinoids are often defined in pharmacological terms as a class of compounds that exceed threshold-binding affinities for specific receptors found in central-nervous-system tissues and/or peripheral tissues. The interactions between cannabinoids and their receptors are under investigation by a number of researchers, because the resultant effects are demonstrably important both in medicinal and reactional contexts. Many medicinal and recreational cannabinoid products feature cannabinoids in crystalline or otherwise solid form, and many methods for producing or extracting cannabinoids yield solid materials. Unfortunately, solid-form cannabinoids are not well suited to some applications and products. For example, many vape devices and a number of manufacturing processes require cannabinoid compositions that are flowable under the relevant conditions. In such instances, diluents are often used to provide sufficient flowability by dissolving or otherwise mobilizing the cannabinoid compositions. However, diluent-based mobilization strategies are often not satisfactory—on one hand, increasing diluent incorporation into a cannabinoid composition reduces the potency of the composition (i.e. the cannabinoid concentration)—on the other hand, decreasing diluent incorporation tends to reduce stability with respect to cannabinoid precipitation, and this may impact flowability. In general, there is an unmet need for cannabinoid compositions that are flowable and that have high-effective concentrations.

### SUMMARY

The experimental results set out in the present disclosure demonstrate that flowability and high effective concentrations are not mutually exclusive in the context of cannabinoid compositions. In particular, the present disclosure asserts that this desirable combination is attainable because flowability is a key feature when a cannabinoid composition is in situ (i.e. in reserve form) and that high effective concentration is a key feature when a cannabinoid composition is in actio (i.e. in delivery form). In this context, the present disclosure asserts that some cannabinoids are capable of acting as “incipients”—substances that increase the in actio concentration of a target component without increasing the in situ concentration of the target component above its saturation concentration.

A cannabinoid composition comprising a 2:1 mixture of cannabidiolic acid (CBDA) and cannabidiol (CBD) and only minor amounts of other products provides a non-limiting illustration of how a cannabinoid incipient can be utilized to increase the in actio concentration of a target cannabinoid without increasing the in situ concentration of the target cannabinoid above its saturation concentration. In this case, cannabinoid compositions comprising high effective concentrations of CBD (e.g. greater than 60 wt. % CBD or greater than 85% CBD) are desirable, but experimental results indicate that, under at least some vape-device related conditions, CBD has a saturation concentration of about 50 wt. %. In other words, under at least some vape-device related conditions, CBD solutions comprising greater than about 50 wt. % CBD may suffer from reduced flowability within a vape device. As such, high effective CBD concentrations appear untenable based on conventional strategies. However, the experimental results set out herein also indicate that: (i) the saturation concentration of CBDA under similar circumstances is considerably higher than that of CBD; (ii) CBDA can be converted to CBD under the conditions associated with cannabinoid vapourization (e.g. the formation of a cannabinoid-based vapour or aerosol); and (iii) including CBDA in an in situ composition can increase the saturation concentration of CBD. Accordingly, as evidenced by the results set out in the present disclosure, CBDA can be utilized as an incipient—it can increase the in actio concentration of CBD without increasing the in situ concentration of CBD above its saturation concentration. For example, the experimental results set out herein indicate that, under select conditions, a 65:32:3 ratio of CBDA to CBD to terpenes (on weight basis) remains flowable within the reservoir of a vape device (i.e. in situ), and yet provides an effective concentration of CBD on vaporization (i.e. in actio) that it is higher than the saturation concentration of CBD within the reservoir. In this respect, the present disclosure provides access to flowable cannabinoid compositions having high effective concentrations.

Select embodiments of the present disclosure relate to a high-effective-concentration cannabinoid-based vape oil composition, comprising: a cannabinoid-based incipient; and a cannabinoid-based target component, wherein: the cannabinoid-based incipient has an in-situ concentration that is less than the saturation concentration of the cannabinoid-based incipient, the cannabinoid-based target component has an in-situ concentration that is less than the saturation concentration of the cannabinoid-based target component, the in-situ concentration of the cannabinoid-based incipient and the in-situ concentration of the cannabinoid-based target component, taken together, is greater than the saturation concentration of the cannabinoid-based target component, yet the cannabinoid-based vape oil is flowable, and the cannabinoid-based target component has an in-actio concentration that is greater than the saturation concentration of the cannabinoid-based target component, such that the cannabinoid-based vape oil has a high-effective concentration of the cannabinoid-based target component.

Select embodiments of the present disclosure relate to a cannabinoid composition, comprising: cannabidiolic acid (CBDA); and cannabidiol (CBD); wherein: the CBDA accounts for between about 20.0 wt. % and about 70.0 wt. % of the composition, the CBD accounts for between 30.0 wt. % and about 50.0 wt % of the composition, and the composition is flowable within a vape device.

Select embodiments of the present disclosure relate to a cannabinoid composition, comprising: a cannabinoid-based incipient; and a cannabinoid-based target component, wherein: the cannabinoid-based incipient has an in-situ concentration that is less than the saturation concentration of the cannabinoid-based incipient and the cannabinoid-based target component has an in-situ concentration that is less than the saturation concentration of the cannabinoid-based target component, such that the cannabinoid composition is flowable, and the in-situ concentration of the cannabinoid-based incipient and the in-situ concentration of the cannabinoid-based target component, taken together, is greater than the saturation concentration of the cannabinoid-based target component.

Select embodiments of the present disclosure relate to a method of preparing a cannabinoid-based vape oil that is formulated for high-effective-concentration vaping, comprising: combining a cannabinoid-based incipient, a cannabinoid-based target component, and a matrix to form a first composition; reducing the relative amount of the matrix in the first composition to form a cannabinoid-based vape oil in which: (i) the cannabinoid-based incipient has a concentration that is less than the saturation concentration of the cannabinoid-based incipient in the vape oil, and (ii) the cannabinoid-based target component has a concentration, that is less than the saturation concentration of the cannabinoid-based target component in the vape oil, wherein the concentration of the cannabinoid-based incipient in the vape oil and the concentration of the cannabinoid-based target component in the vape oil, taken together, is greater than the saturation concentration of the cannabinoid-based target component in the vape oil, such that the vape oil is formulated for high-effective concentration vaping.

Select embodiments of the present disclosure relate to a vape device, comprising: a payload reservoir that is loaded with a cannabinoid composition comprising: (i) a cannabinoid-based incipient at an in-situ concentration that is less than the saturation concentration of the cannabinoid-based incipient, and (ii) a cannabinoid-based target component at an in-situ concentration that is less than the saturation concentration of the cannabinoid-based target component, wherein the in-situ concentration of the cannabinoid-based target component, taken together, is greater than the saturation concentration of the cannabinoid-based target component; a vapourizing element configured to: (i) vapourize at least a portion of the cannabinoid-based target component, and (ii) convert at least a portion of the cannabinoid-based incipient into the cannabinoid-based target component, such that the cannabinoid-based target component has an in-actio concentration that is greater than the in-situ saturation concentration of the cannabinoid-based target component; and an inhalation aperture, configured to allow a user to inhale at least a portion of the cannabinoid composition after at least a portion of the cannabinoid composition is exposed to the vapourizing element.

These and other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 show a schematic phase diagram for a cannabinoid composition in accordance with the present disclosure that comprises CBD derived from an isolate and CBDA.

FIG. 2 shows a flow chart of method steps for preparing a cannabinoid-based composition that is flowable and that has a high effective concentration.

FIG. 3 shows a high-performance liquid chromatography with diode array detection (HPLC-DAD) chromatogram of a composition comprising CBDA and CBD derived from an isolate in a 1:1.31 CBDA:CBD ratio.

FIG. 4 shows an HPLC-DAD chromatogram of a composition comprising CBDA and CBD derived from an isolate in a 1.18:1 CBDA:CBD ratio.

### DETAILED DESCRIPTION

Embodiments of the present disclosure will now be described with reference to the accompanying drawings.

As noted above, there is an unmet need for cannabinoid compositions that are flowable and that have high effective concentrations. For example, many vape devices and a number of manufacturing processes require cannabinoid compositions that are movable by injection, wicking (i.e. capillary action), gravity, and/or pumping. Such compositions have the additional benefit that they may be compatible with volumetric-dosing techniques as used in some continuous manufacturing processes and state-of-the-art vape devices. In the context of the present disclosure, a cannabinoid composition is flowable if its movement through a vape device or manufacturing process is not substantially impeded by precipitation, crystallization, solidification, and/or deposition from the cannabinoid composition. In the context of the present disclosure, a cannabinoid composition has a high effective concentration if the in actio concentration of a cannabinoid component is greater than the in situ saturation concentration of the cannabinoid component.

The experimental results set out in the present disclosure demonstrate that flowability and high effective concentrations are not mutually exclusive in the context of cannabinoid compositions. In particular, the present disclosure asserts that this desirable combination is attainable because flowability is a key feature when a cannabinoid composition is in situ (i.e. in reserve form) and that high effective concentration is a key feature when a cannabinoid composition is in actio (i.e. in delivery form). In this context, the present disclosure asserts that some cannabinoids are capable of acting as “incipients”—substances that increase the in actio concentration of a target component without increasing the in situ concentration of the target component above its saturation concentration.

As used herein, the term “cannabinoid” refers to: (i) a chemical compound belonging to a class of secondary compounds commonly found in plants of genus cannabis; and/or (ii) one of a class of diverse chemical compounds that may act on cannabinoid receptors such as CB1 and CB2.

In an embodiment, the cannabinoid is a compound found in a plant, e.g., a plant of genus cannabis, and is sometimes referred to as a phytocannabinoid. In one embodiment, the cannabinoid is a compound found in a mammal, sometimes called an endocannabinoid. In one embodiment, the cannabinoid may be made in a laboratory setting, sometimes called a synthetic cannabinoid. In one embodiment, the cannabinoid may be derived or obtained from a natural source (e.g. plant) but is subsequently modified or derivatized in one or more different ways in a laboratory setting, sometimes called a semi-synthetic cannabinoid.

Tetrahydrocannabinol (THC) is the primary psychoactive compound in cannabis and one of the most notable cannabinoids of the phytocannabinoids. Cannabidiol (CBD) is another cannabinoid that is a major constituent of the phytocannabinoids. There are at least 113 different cannabinoids in cannabis, and each can exhibit varied pharmacologic and physiologic effects.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to: cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic Acid (CBCA), cannabichromene (CBC), cannabichromevarinic Acid (CBCVA), cannabichromevarin (CBCV), cannabidiolic acid (CBDA), cannabidiol (CBD), Δ6-cannabidiol (Δ6-CBD), cannabidiol monomethylether (CBDM), cannabidiol-C4 (CBD-C4), cannabidivarinic Acid (CBDVA), cannabidivarin (CBDV), cannabidiorcol (CBD-C1), tetrahydrocannabinolic acid A (THCA-A), tetrahydrocannabinolic acid B (THCA-B), tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-α10-THC), tetrahydrocannabinolic acid C4 (THCA-C4), tetrahydrocannbinol C4 (THC-C4), tetrahydrocannabivarinic acid (THCVA), tetrahydrocannabivarin (THCV), Δ8-tetrahydrocannabivarin (Δ8-THCV), Δ9-tetrahydrocannabivarin (Δ9-THCV), tetrahydrocannabiorcolic acid (THCA-C1), tetrahydrocannabiorcol (THC-C1), Δ7-cis-iso -tetrahydrocannabivarin, Δ8-tetrahydrocannabinolic acid (Δ8-THCA), Δ9-tetrahydrocannabinolic acid (Δ9-THCA), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabino-C2 (CBN-C2), cannabiorcol (CBN-C1), cannabinodiol (CBND), cannabinodivarin (CBDV), cannabitriol (CBT), 11-hydroxy-Δ9-tetrahydrocannabinol (11-OH-THC), 11 nor 9-carboxy-δ9-tetrahydrocannabinol, ethoxy-cannabitriolvarin (CBTVE), 10 ethoxy-9-hydroxy-δ6a -tetrahydrocannabinol, cannabitriolvarin (CBTV), 8,9 dihydroxy-Δ6a(10a) -tetrahydrocannabinol (8,9-Di-OH-CBT-C5), dehydrocannabifuran (DCBF), cannbifuran (CBF), cannabichromanon (CBCN), cannabicitran, 10 oxo-Δ6a(10a)-tetrahydrocannabinol (OTHC), Δ9-cis-tetrahydrocannabinol (cis-THC), cannabiripsol (cbr), 3,4,5,6-tetrahydro-7-hydroxy-alpha-alpha-2-trimethyl-9-n-propyl-2,6-methano-2h-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, hexahydrocannibinol, and dodeca-2e,4e-dienoic acid isobutylamide.

In some embodiments of the present disclosure, the cannabinoid is a cannabinoid dimer. The cannabinoid may be a dimer of the same cannabinoid (e.g. THC—THC) or different cannabinoids. In an embodiment of the present disclosure, the cannabinoid may be a dimer of THC, including for example cannabisol.

As used herein, the term “THC” refers to tetrahydrocannabinol. “THC” is used interchangeably herein with “Δ9-THC”.

Structural formulae of cannabinoids of the present disclosure may include the followingCBDA:

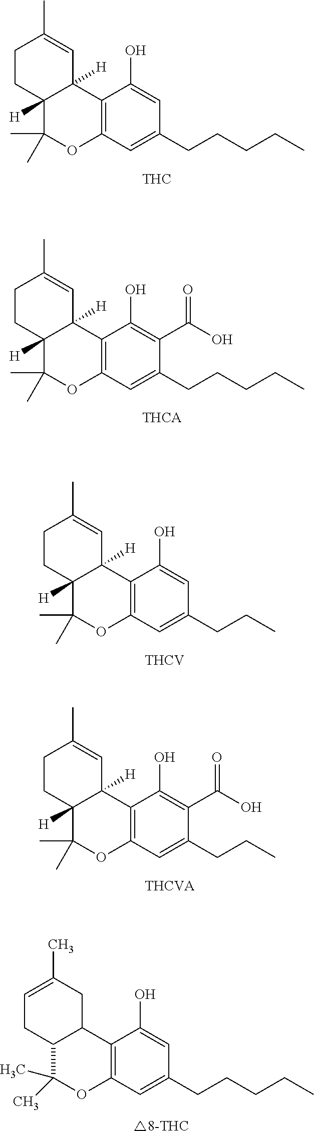

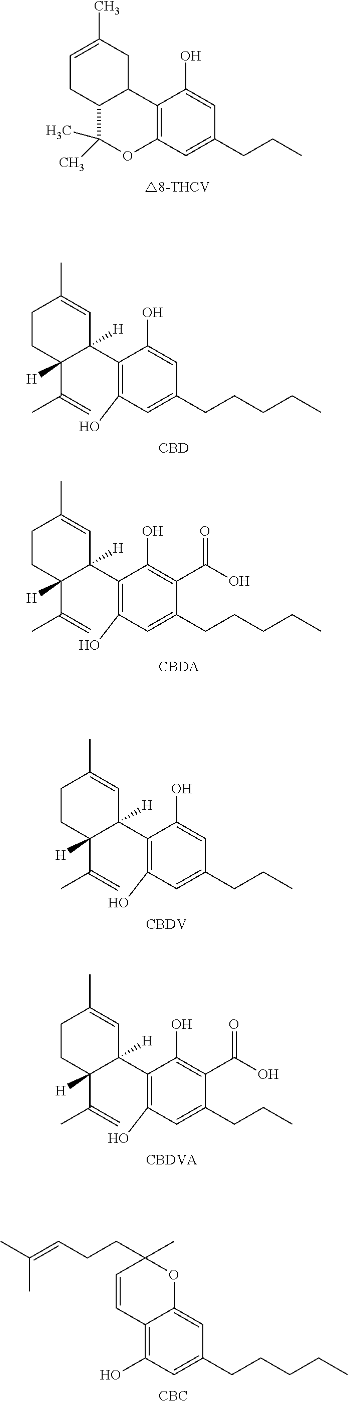

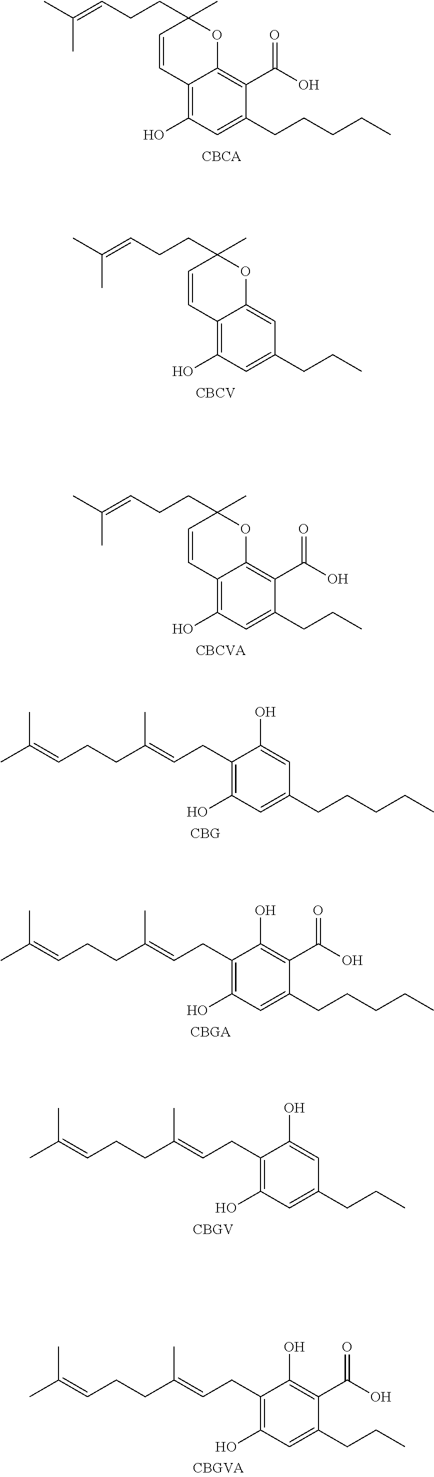

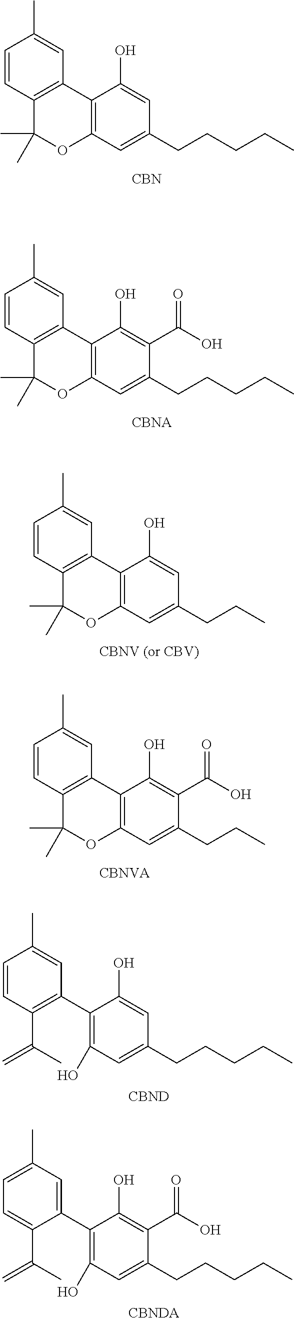

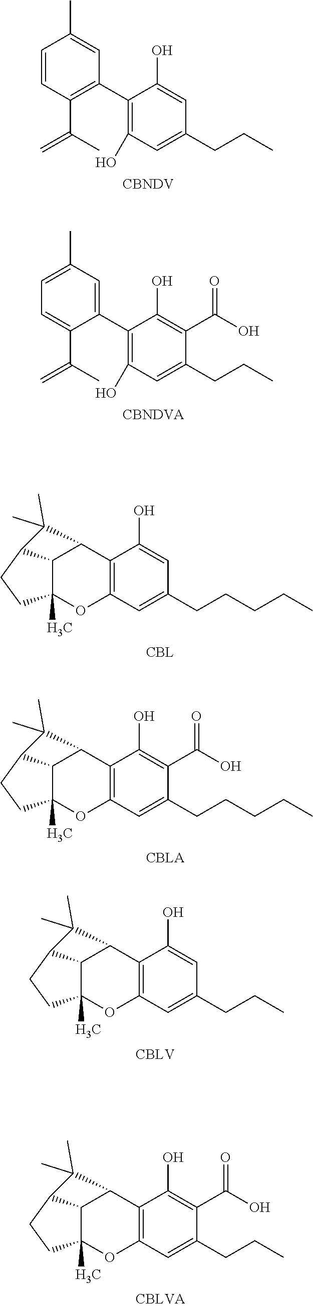

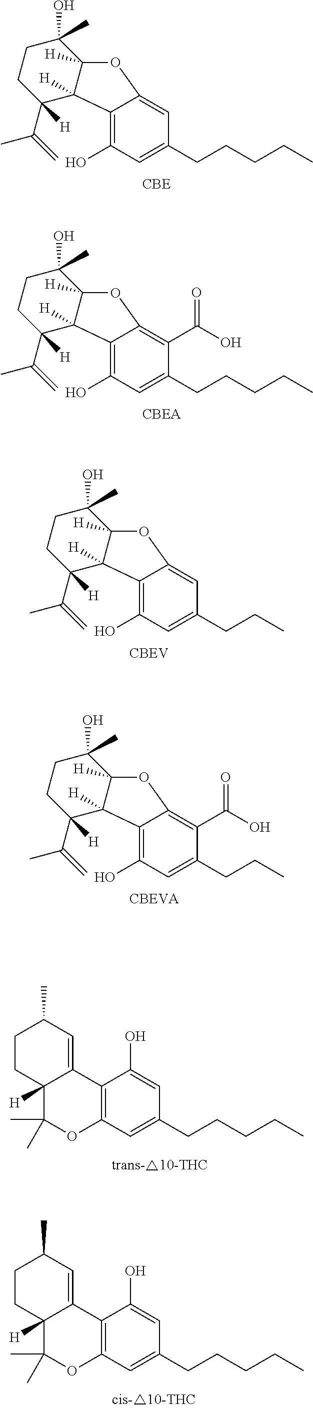

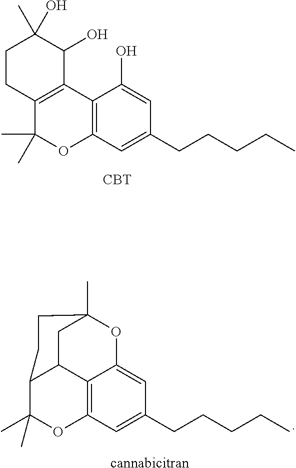

In select embodiments of the present disclosure, the cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

Those skilled in the art who have benefited from the teachings of the present disclosure will recognize that the cannabinoids listed above may have additional acid forms to those depicted. Moreover, those skilled in the art who have benefited from the teachings of the present disclosure will recognize that acid-form cannabinoids and/or neutral-form cannabinoids may be derivatized, such as to form an ester and/or sulfonic ester derivative of one or more of the cannabinoids identified above without falling outside the scope of the present disclosure.

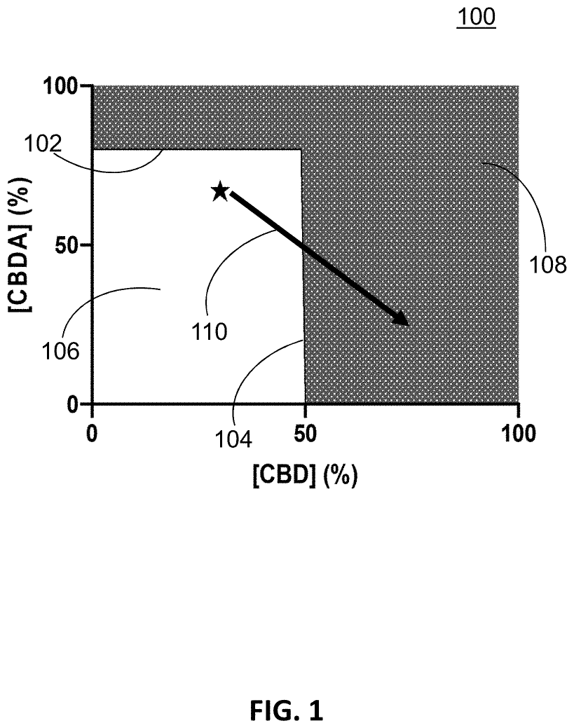

FIG. 1 illustrates this with a schematic phase diagram 100 for a cannabinoid composition in accordance with the present disclosure that comprises CBDA and CBD. Experimental results indicate that CBDA and CBD have saturation concentrations of about 70 wt. % and about 50 wt. % under a first set of test conditions (i.e. ethanol solutions under standard temperature and pressure conditions, wherein the CBD is derived from an isolate). In FIG. 1, the saturation concentration of CBDA and CBD are identified with reference numbers 102 and 104, respectively, and they delineate a “potentially flowable zone” 106 from a “non-flowable zone” 108. Experimental results indicate that increasing the in situ CBD concentration of a CBDA/CBD mixture beyond its saturation concentration 104 results in cannabinoid precipitation and therefore non-flowable cannabinoid compositions. Likewise, experimental results indicate that increasing the in situ CBDA concentration of a CBDA/CBD mixture beyond its saturation concentration 102 results in cannabinoid precipitation and therefore non-flowable cannabinoid compositions. Importantly, however, experimental results also indicate that some CBDA/CBD mixtures have CBDA/CBD concentrations that are below the CBDA/CBD saturation concentrations in situ such that they fall within the potentially flowable zone 106 and do not crystalize under the evaluation conditions, yet they have in-actio CBD concentrations that are above the CBD saturation concentration 104 due to the conversion of CBDA to CBD during delivery (e.g. vapourization). This is indicated schematically in FIG. 1 with arrow 110. Arrow 110 highlights an experimental result in which a cannabinoid composition having an in situ CBD concentration that is less than the CBD saturation concentration 104 is converted into a composition having an in actio CBD concentration that is greater than the CBD saturation concentration 104, because CBDA converted to CBD in actio. In other words, the experimental result exemplifies the potential for CBDA to act as a CBD incipient to provide a flowable cannabinoid composition that has a high effective concentration of CBD.

More generally, select embodiments of the present disclosure relate to a cannabinoid composition, comprising: a cannabinoid-based incipient and a cannabinoid-based target component, wherein: the cannabinoid-based incipient has an in-situ concentration that is less than the saturation concentration of the cannabinoid-based incipient and the cannabinoid-based target component has an in-situ concentration that is less than the saturation concentration of the cannabinoid-based target component, such that the cannabinoid composition is flowable, and the in-situ concentration of the cannabinoid-based incipient and the in-situ concentration of the cannabinoid-based target component, taken together, is greater than the saturation concentration of the cannabinoid-based target component.

In select embodiments of the present disclosure, the cannabinoid-based target component may be a neutral-form cannabinoid. In select embodiments of the present disclosure, the cannabinoid-based target component may be an acid-form cannabinoid. In select embodiments of the present disclosure, the cannabinoid-based incipient may be a neutral-form cannabinoid. In select embodiments of the present disclosure, the cannabinoid-based incipient may be an acid-form cannabinoid.

In the compositions of the present disclosure, the ratio of the cannabinoid-based incipient to the cannabinoid-based target component (in situ) may vary. For example, the ratio of the cannabinoid-based incipient to the cannabinoid-based target component may be between about 3.0:1.0 and about 1.0:1.0. In particular, the ratio of the cannabinoid-based incipient to the cannabinoid-based target component may be between about 1.6:1,0 and about 1.0:1.0. Alternatively, the ratio of the cannabinoid-based incipient to the cannabinoid-based target component may be between about 10.0:1.0 and about 3.0:1.0. Alternatively, the ratio of the cannabinoid-based incipient to the cannabinoid-based target component may be between about 1.0:1.0 and about 1.0:10.0. Suitable ratios may be selected in view of the teachings of the present disclosure. By way of non-limiting example, EXAMPLE 1 provides framework for determining a suitable ratio for an incipient/target compound pair based on CBDA and CBD under a specific set of conditions wherein the CBD is derived from an isolate. Of course, determining a suitable ratio for any particular incipient/target component pair will depend on a variety of factors (e.g. temperature, concentrations, matrix composition, time, etc.). For example, the high-effective-concentration cannabinoid compositions of the present disclosure may further comprise cannabinoid-based additives, such as cannabigerol (CBG). The results of the present disclosure suggest that, under select conditions, significant concentrations of CBG may be included in select high-effective-concentration cannabinoid compositions while maintaining the in-situ concentrations of the cannabinoid-based incipient and the cannabinoid-based target component below their respective saturation concentrations. In the context of CBD/CBDA compositions, without being bound to any particular theory, CBG may increase the saturation concentration of CBD and/or CBDA by doping effects, dimerization, altering colligative properties, and/or increasing crystal lattice energy states.

In select embodiments of the present disclosure, the cannabinoid-based incipient may be convertible into the cannabinoid-based target component by decarboxylation. For example, the cannabinoid-based incipient may be CBDA and the cannabinoid-based target component may be CBD. Alternatively, the cannabinoid-based incipient may be convertible into the cannabinoid-based target component by isomerization oxidation, or other types of chemical transformations.

In select embodiments of the present disclosure, the cannabinoid composition may have a viscosity that is less than about 80,000 cP under standard temperature and pressure conditions. For example, the cannabinoid composition may have a viscosity that is between about 25 cP and about 65,000 cP under standard temperature and pressure conditions, in particular between 40 cP and 60,000 cP under standard temperature pressure and temperature conditions. More generally, the cannabinoid composition may have a viscosity that ensures flowability within a vape device or within a manufacturing process. Those skilled in the art having benefitted from the teachings of the present disclosure will recognize the conditions typically associated with fluid-transport within a vape device or a manufacturing process.

In select embodiments of the present disclosure, the in-situ concentration of the cannabinoid-based incipient and the in-situ concentration of the cannabinoid-based target component, taken together, may be at least 20% greater than the saturation concentration of the cannabinoid-based target component. For example the in-situ concentration of the cannabinoid-based incipient cannabinoid-based target component, taken together, may be at least 30%, 40%, or 50%, greater than the saturation concentration of the cannabinoid- based target component.

In select embodiments of the present disclosure, the cannabinoid-based incipient, the cannabinoid-based target component, or a combination thereof may comprise a pure compound, a distillate, an extract, and/or an isolate. As a first example, the cannabinoid-based incipient and the cannabinoid-based target component may each be obtained from a hemp extract or a marijuana extract by methods known to those skilled in the art. As a second example, the cannabinoid-based target component may be derived from an isolate, and the cannabinoid-based incipient may be derived from an extract. As a third example, the cannabinoid-based target component may be derived from a distillate, and the cannabinoid-based incipient may be derived from an extract. In select embodiments of the present disclosure, the cannabinoid-based target component is CBD derived from an isolate and the cannabinoid-based incipient is chromatographically purified CBDA. The chromatographically purified CBDA may be derived from non-decarboxylated extract. Purified CBDA may comprise other cannabinoids in small quantities.

Importantly, the saturation concentration of any particular cannabinoid-based target component and/or any particular cannabinoid-based incipient may be a function of composition. For example, CBD distillates may yield higher saturation concentrations than CBD isolates. Without being bound to any particular theory, CBG may increase the saturation concentration of CBD and/or CBDA by doping effects, dimerization, altering colligative properties, and/or increasing crystal lattice energy states.

In select embodiments of the present disclosure, the high-effective-concentration cannabinoid compositions comprise CBG as a cannabinoid-based additive. In select embodiments of the present disclosure, the CBG accounts for: (i) between about 0.5% and about 10% of the composition; (ii) between about 10% and about 20% of the composition; or (iii) between about 20% and about 30% of the composition.

Select embodiments of the present disclosure relate to a high-effective-concentration cannabinoid-based vape oil that is flowable within a vape device. The high-effective-concentration cannabinoid-based vape oil comprises a cannabinoid-based incipient and a cannabinoid-based target component. The cannabinoid-based incipient has an in-situ concentration that is less than the saturation concentration of the cannabinoid-based incipient. The cannabinoid-based target component has an in-situ concentration that is less than the saturation concentration of the cannabinoid-based target component. The in-situ concentration of the cannabinoid-based incipient and the in-situ concentration of the cannabinoid-based target component, taken together, is greater than the saturation concentration of the cannabinoid-based target component. The cannabinoid-based target component has an in-actio concentration that is greater than the saturation concentration of the cannabinoid-based target component. As such, the cannabinoid-based vape oil has a high-effective concentration of the cannabinoid-based target component.

In select embodiments of the present disclosure, the in-actio concentration of the cannabinoid-based target component may be at least 20% greater than the saturation concentration of the cannabinoid-based target component. For example the in-actio concentration of the cannabinoid-based target component, may be at least 30%, 40%, or 50%, greater than the saturation concentration of the cannabinoid-based target component.

Select embodiments of the present disclosure relate to a cannabinoid composition comprising cannabidiolic acid (CBDA) and cannabidiol (CBD). The CBDA accounts for between about 20.0 wt. % and about 70.0 wt. % of the composition, the CBD accounts for at least about 30.0 wt. % and about 50.0 wt. % of the composition, and the composition is flowable within a vape device.

Select embodiments of the present disclosure relate to a method of preparing a cannabinoid-based vape oil that is formulated for high-effective-concentration vaping. The method comprises combining a cannabinoid-based incipient, a cannabinoid-based target component, and a matrix to form a first composition. The method further comprises reducing the relative amount of the matrix in the first composition to form the cannabinoid-based vape oil. With respect to the cannabinoid-based vape oil: (i) the cannabinoid-based incipient has a concentration that is less than the saturation concentration of the cannabinoid-based incipient in the vape oil; and (ii) the cannabinoid-based target component has a concentration that is less than the saturation concentration of the cannabinoid-based target component in the vape oil. The concentration of the cannabinoid-based incipient in the vape oil and the cannabinoid-based target component in the vape oil, taken together, is greater than the saturation concentration of the cannabinoid-based target component in the vape oil, such that the vape oil is formulated for high-effective concentration vaping.

In select embodiments of the present disclosure, the reducing of the relative amount of the matrix in the first composition may be executed using a vacuum, a heating device, or a combination thereof. Of course, the reducing of the relative amount of the matrix in the first composition may be executed by simple evaporation.

In select embodiments of the present disclosure, the matrix may comprise a class III solvent. For example, the matrix may comprise ethanol, heptane, or a combination thereof. More generally, the matrix may comprise a solvent such as pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

In select embodiments of the present disclosure, a cannabinoid composition may further comprise a terpene. In the context of the present disclosure, a terpene is a compound built on an isoprenoid structure or produced by combining isoprene units, which comprise five-carbon structures. In select embodiments of the present disclosure, the terpene is a hydrocarbon. In the context of this disclosure, the term “terpene” does not necessarily require five carbons or multiples of five carbons. Those skilled in the will appreciate that a reaction with isoprene units does not always result in a terpene comprising all the carbon atoms. In the context of this disclosure, the term “terpene” includes cannabis-derived terpenes and non-cannabis derived terpenes. In the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, combinations thereof, and derivatives thereof. Likewise, in the context of this disclosure, the term “terpene” includes isomeric, enantiomeric, or optically active derivatives. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. In the context of the present disclosure, the term “terpene” includes the a (alpha), β- (beta), γ- (gamma), oxo -,isomers, or any combinations thereof. Examples of terpenes within the context of this disclosure include, without limitation: 7,8 dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta -3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta -Eudesmol) (Gamma-Eudesmol), Eugenol, Euphol, Farnesene, Farnesol, Fenchol (Beta -Fenchol), Fenchone, Geraniol, Geranyl acetate, Germacrenes, Germacrene B, Guaia -1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

Select embodiments of the present disclosure relate to a vape device that comprises a reservoir, a vapourizing element, and an inhalation orifice. The reservoir, the vapourizing element, and the inhalation orifice are fluidically coupled. The reservoir houses a cannabinoid composition comprising a cannabinoid-based incipient and a cannabinoid-based target component. The cannabinoid-based incipient has an in-situ concentration that is less than the saturation concentration of the cannabinoid-based incipient. The cannabinoid-based target component has an in-situ concentration that is less than the saturation concentration of the cannabinoid-based target component. The in-situ concentration of the cannabinoid-based incipient and the in-situ concentration of the cannabinoid-based target component, taken together, is greater than the saturation concentration of the cannabinoid-based target component. The vapourizing element is configured to: (i) vapourize at least a portion of the cannabinoid-based target component; and (ii) convert at least a potion of the cannabinoid-based incipient into the cannabinoid-based target component, such that the cannabinoid-based target component has an in-actio concentration that is greater than the in-situ saturation concentration of the cannabinoid-based target component.

### EXAMPLES

In addition to the experimental results summarized with reference to FIG. 1 above, further experimental results indicate that careful selection of the ratio of a cannabinoid-based incipient to a cannabinoid-based target component facilitates the preparation of flowable, high effective concentration cannabinoid compositions. For example, a first set of experiments was completed using CBD derived from an isolate and CBDA to determine which high effective concentration CBDA/CBD ratios remain flowable over a time as set out in EXAMPLES 1-3. A second set of experiments was completed using CBD derived from a distillate and CBDA to determine which high effective concentration CBDA/CBD ratios remain flowable over a time as set out in EXAMPLES 4. A third set of experiments was completed to evaluate the stability of CBDA/CBD compositions in the presence of cannabigerol (CBG) as a cannabinoid-based additive as set in EXAMPLES 5-6.

### EXAMPLE 1

In this example, CBD isolate and CBDA were used to prepare cannabinoid compositions of varying CBDA/CBD ratios (ratios derived using the masses of the oils) in accordance with a method of the present disclosure and loaded into vape reservoirs (i.e. cartridges configured for use in vape devices). In particular, the cannabinoid compositions were triturated in ˜5 mL of ethanol, and then the ethanol concentration was reduced by agitation at room temperature until the concentration fell below 1,000 ppm. The compositions were then infused with about 3% terpenes by mass, and volumes were transferred to vape cartridges for loading into vape devices. The vape devices comprised C-cell brand TH2 cartridges with 0.5 mL glass tanks, ceramic mouthpieces, 2 mm apertures, ceramic wicks, dual-coil designs, and pressure-sensitive componentry.

The CBD isolate/CBDA cannabinoid compositions were evaluated over time to determine the extent to which they remained flowable in situ. Results are indicated in TABLE 1.

TABLE 1
Experimental results from evaluating a series of high effective
concentration cannabinoid compositions having various cannabinoid-
based incipient/cannabinoid-based target component ratios and their
tendency to retain flowability over time. CBDA:CBD ratios derived from
the masses of oils used to prepare the composition.

| | CBDA:CBD | |
|---|---|---|
| Identifier | isolate ratio | Experimental observations on flowability |
| A | 1.0:9.0 | Loss of flowability within one day |
| B | 1.0:4.0 | Loss of flowability within one day |
| C | 1.0:2.0 | Loss of flowability within one week |
| D | 1.0:1.0 | Loss of flowability between 1 and 2 weeks |
| E | 2.0:1.0 | No loss of flowability after at least 6 months |
| F | 4.0:1.0 | Loss of flowability between 3 and 5 weeks |

Importantly, composition E exhibited no loss of flowability over a 5-month period. To further evaluate this result, the 2.0:1.0 CBDA:CBD isolate composition was scaled up and stress tested under favourable crystallization conditions as set out in EXAMPLE 2.

### EXAMPLE 2

A set of vape reservoirs were charged with 500 mg of a 2.0:1.0 CBDA:CBD distillate composition prepared in accordance with the protocol described in EXAMPLE 1. A first sub-set of the charged vape reservoirs were evaluated over a period of about six weeks without further manipulation, and no loss of flowability was observed. A second sub-set of the charged vape reservoirs were vaped to completion over a period of about six weeks, and no loss of flowability was observed. A third sub-set of the charged vape reservoirs were “micro-seeded” with various solid compositions to evaluate the flowability of the composition in the presence of potential crystallization-inducing materials. Results from the micro-seeded charged vape reservoirs are summarized in TABLE 2.

TABLE 2
Experimental results from a series of high effective concentration
cannabinoid compositions having 2.0:1.0 cannabinoid-based
incipient/cannabinoid-based target component ratios stress tested by
addition of various micro-seeds.

| Type of micro-seed | Comments on flowability over time |
|---|---|
| CBD isolate | No loss of flowability |
| CBDA isolate | No loss of flowability |
| Both CBD and CBDA isolate | No loss of flowability |

Importantly, no loss of flowability was observed in the presence of all three types of micro-seeds.

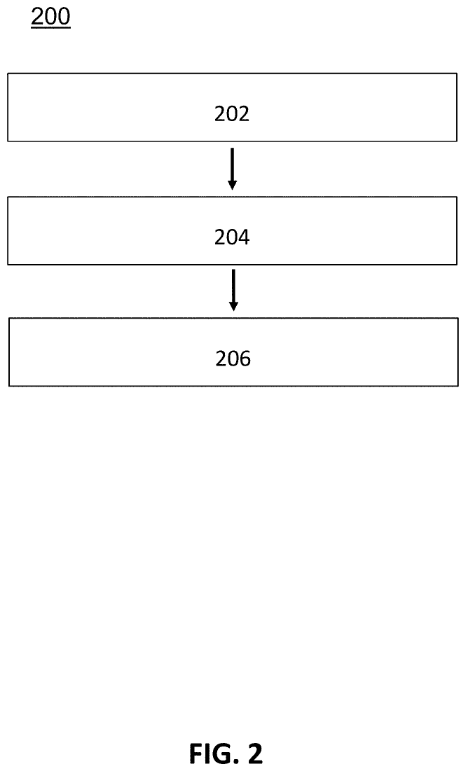

FIG. 2 provides a flow chart 200 of method steps used to prepare the CBDA/CBD compositions of EXAMPLE 1 and EXAMPLE 2. The steps set out in FIG. 2 are provided by way of example only and are not limiting on the scope of the present disclosure.

The flow chart 200 comprises a step 202 of combining about 8.8 g of CBDA, about 4.0 g of CBD isolate, and about 10 mL of ethanol to form a first composition under standard temperature/pressure conditions. The flow chart 200 further comprises a step 204 of removing at least a portion of the ethanol in the first composition by evaporation in a fume hood to provide a cannabinoid-based vape oil (for example having less than 1,000 ppm ethanol by weight). The flow chart 200 comprises a step 206 of homogenizing terpenes into the oil to provide a composition comprising about 67% CBDA, about 30% CBD, and about 3% terpenes (by weight).

### EXAMPLE 3

A series of fine-ratio experiments were performed to study the effect of CBDA:CBD isolate ratio on composition stability; i.e. whether they remained flowable in situ (stable) or crystallized. For these experiments, the quantities of CBDA and CBD in the compositions were analytically determined, rather than derived from the masses of the oils used. The ratios investigated are summarized in TABLE 3.

TABLE 3
Experimental results after about 4 weeks from a series of high
effective concentration cannabinoid compositions having various
cannabinoid-based incipient/cannabinoid-based target component ratios.
CBDA and CBD quantities determined analytically.

| | CBDA:CBD | |
|---|---|---|
| Identifier | isolate ratio | Experimental observations |
| A | 1.0:1.34 | Crystallized |
| B | 1.0:1.11 | Crystallized |
| C | 1.06:1.0 | Stable |
| D | 1.18:1.0 | Stable |
| E | 1.31:1.0 | Stable |
| F | 1.54:1.0 | Stable |
| G | 1.71:1.0 | Crystallized |
| H | 1.90:1.0 | Crystallized |
| I | 2.11:1.0 | Crystallized |

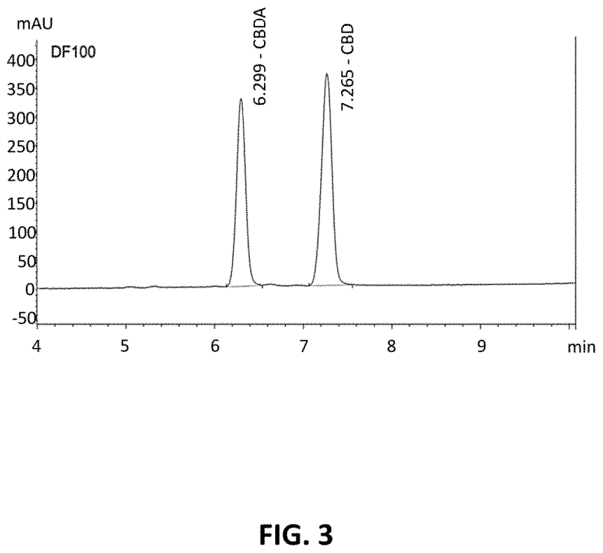

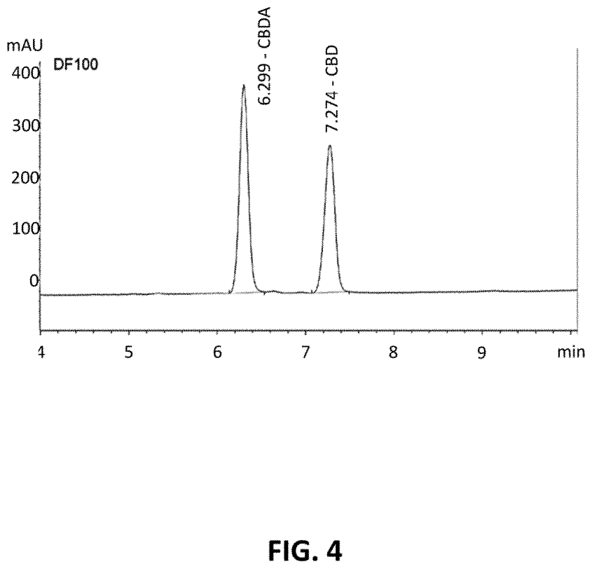

The compositions were prepared in accordance with the protocol described in EXAMPLE 1. In experiment A, a set of vape reservoirs were charged with 51.4 mg of a 1.0:1.34 CBDA:CBD composition. The charged vape reservoirs were evaluated over a period of about four weeks without further manipulation and crystallization was observed. FIG. 3 shows an HPLC-DAD chromatogram of the composition after about four weeks. In experiment D, a set of vape reservoirs were charged with 51.4 mg of a 1.18:1 CBDA:CBD composition. The charged vape reservoirs were evaluated over a period of about four weeks without further manipulation. No crystallization was observed and the composition remained flowable. FIG. 4 shows an HPLC-DAD chromatogram of the composition after about 4 weeks. After about 7.5 months standing at room temperature, experiments C and D continued to be stable but some crystallization was observed in experiments E and F.

### EXAMPLE 4

In this example, cannabinoid compositions of varying CBDA/CBD ratios (ratios determined analytically) were prepared in accordance with a method of the present disclosure and loaded into vape reservoirs (i.e. cartridges configured for use in vape devices). The compositions were derived from CBD distillate and CBDA. The cannabinoid compositions were triturated in ˜5 mL of ethanol, and then the ethanol concentration was reduced by agitation at room temperature until the concentration fell below 1,000 ppm. The compositions were then infused with about 3% terpenes by mass, and volumes were transferred to vape cartridges for loading into vape devices. The vape devices comprised C-cell brand TH2 cartridges with 0.5 mL glass tanks, ceramic mouthpieces, 2 mm apertures, ceramic wicks, dual-coil designs, and pressure-sensitive componentry.

The cannabinoid compositions were evaluated over time to determine the extent to which they remained flowable in situ. Results are indicated in TABLE 4.

TABLE 4
Experimental results from evaluating a series of high effective
concentration cannabinoid compositions having various cannabinoid-
based incipient/cannabinoid-based target component ratios and their
tendency to retain flowability over time. CBDA:CBD ratios were
determined analytically.

| | | | Experimental |
|---|---|---|---|
| | CBDA:CBD | Cannabinoid content | observations on |
| Identifier | distillate ratio | as % of composition | flowability |
| A | 1.0:5.5 | Total cannabinoids = 87.1% | No loss of |
| | | CBD = 72.8% | flowability after |
| | | CBDA = 13.1% | about 11 weeks |
| B | 1.0:2.4 | Total cannabinoids = 90.3% | No loss of |
| | | CBD = 63.0% | flowability after |
| | | CBDA = 26.2% | about 11 weeks |
| C | 1.0:1.3 | Total cannabinoids = 92.0% | No loss of |
| | | CBD = 50.5% | flowability after |
| | | CBDA = 40.3% | about 11 weeks |
| D | 1.4:1.0 | Total cannabinoids = 89.3% | No loss of |
| | | CBD = 36.3% | flowability after |
| | | CBDA = 52.0% | about 11 weeks |
| E | 2.5:1.0 | Total cannabinoids = 88.2% | No loss of |
| | | CBD = 25.1% | flowability after |
| | | CBDA = 62.2% | about 11 weeks |

Importantly, compositions A-E exhibited no loss of flowability over a period of about 11 weeks in spite of the high cannabinoid concentrations.

### EXAMPLE 5

In this example, cannabinoid compositions with consistent CBDA/CBD ratios (ratios determined analytically) were prepared in accordance with a method of the present disclosure and loaded into vape reservoirs (i.e. cartridges configured for use in vape devices) with high effective concentrations. The compositions were derived from CBD distillate and CBDA. The cannabinoid compositions were prepared with varying amounts of cannabigerol (CBG) to evaluate the flowability in the presence of a cannabinoid-based additive. The cannabinoid compositions were triturated in ˜5 mL of ethanol, and then the ethanol concentration was reduced by agitation at room temperature until the concentration fell below 1,000 ppm. The compositions were then infused with about 5% terpenes by mass, and volumes were transferred to vape cartridges for loading into vape devices. The vape devices comprised C-cell brand TH2 cartridges with 0.5 mL glass tanks, ceramic mouthpieces, 2 mm apertures, ceramic wicks, dual-coil designs, and pressure-sensitive componentry.

The cannabinoid compositions were evaluated over time to determine the extent to which they remained flowable in situ. Results are indicated in TABLE 5.

TABLE 5
Experimental results from evaluating a series of high effective
concentration cannabinoid compositions having various cannabinoid-
based incipient/cannabinoid-based target component ratios and their
tendency to retain flowability over time. CBDA:CBD:CBG ratios
were determined analytically.

| | | | Experimental |
|---|---|---|---|
| | CBDA:CBD | Cannabinoid content | observations on |
| Identifier | ratio | as % of composition | flowability |
| A | 1.2:1 | CBD = 35.0% | No loss of |
| | | CBDA = 46.3% | flowability after |
| | | CBG = 0.0% | about 3 months |
| B | 1.2:1 | CBD = 33.7% | No loss of |
| | | CBDA = 44.0% | flowability after |
| | | CBG = 5.2% | about 3 months |
| C | 1.2:1 | CBD = 31.1% | No loss of |
| | | CBDA = 40.6% | flowability after |
| | | CBG = 9.0% | about 3 months |
| D | 1.2:1 | CBD = 28.9% | No loss of |
| | | CBDA = 37.6% | flowability after |
| | | CBG = 16.1% | about 3 months |
| E | 1.2:1 | CBD = 27.4% | No loss of |
| | | CBDA = 36.0% | flowability after |
| | | CBG = 22.4% | about 3 months |

Importantly, the cannabinoid compositions exhibited no loss of flowability over a period of about 3 months while retaining the various concentrations of the cannabinoid-based additive.

### EXAMPLE 6

In this example, cannabinoid compositions with variable CBDA/CBD ratios (ratios determined analytically) were prepared in accordance with a method of the present disclosure and loaded into vape reservoirs (i.e. cartridges configured for use in vape devices) with high effective concentrations. The compositions were derived from CBD distillate and CBDA. The cannabinoid compositions were prepared with varying amounts of cannabigerol (CBG) to evaluate the potential for the compositions of the present disclosure to accommodate additional cannabinoid loads while retaining flowability. The cannabinoid compositions were triturated in ˜5 mL of ethanol, and then the ethanol concentration was reduced by agitation at room temperature until the concentration fell below 1,000 ppm. The compositions were then infused with about 5% terpenes by mass, and volumes were transferred to vape cartridges for loading into vape devices. The vape devices comprised C-cell brand TH2 cartridges with 0.5 mL glass tanks, ceramic mouthpieces, 2 mm apertures, ceramic wicks, dual-coil designs, and pressure-sensitive componentry.

The cannabinoid compositions were evaluated over time to determine the extent to which they remained flowable in situ. Results are indicated in TABLE 6.

TABLE 6
Experimental results from evaluating a series of high effective
concentration cannabinoid compositions having various cannabinoid-
based incipient/cannabinoid-based target component ratios and
various CBG loadings with respect to their tendency to retain
flowability over time.

| | | | Experimental |
|---|---|---|---|
| | CBDA:CBD | Cannabinoid content | observations on |
| Identifier | ratio | as % of composition | flowability |
| A | 1.3:1 | CBD = 34.3% | No loss of |
| | | CBDA = 45.1% | flowability after |
| | | CBG = 0.4% | about 3 months |
| B | 1.2:1 | CBD = 33.4% | No loss of |
| | | CBDA = 41.6% | flowability after |
| | | CBG = 4.8% | about 3 months |
| C | 1.2:1 | CBD = 34.4% | No loss of |
| | | CBDA = 39.8% | flowability after |
| | | CBG = 9.6% | about 3 months |
| D | 1.0:1.0 | CBD = 33.8% | No loss of |
| | | CBDA = 34.1% | flowability after |
| | | CBG = 19.1% | about 3 months |
| E | 1.0:1.2 | CBD = 27.3% | No loss of |
| | | CBDA = 32.5% | flowability after |
| | | CBG = 28.2% | about 3 months |

Importantly, the cannabinoid compositions exhibited no loss of flowability over a period of about 3 months while retaining the various concentrations of the cannabinoid-based additive.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1-48.(canceled)

49. A high-effective-concentration cannabinoid-based vape oil composition, comprising:
a cannabinoid-based incipient; and
a cannabinoid-based target component,
wherein:
the cannabinoid-based incipient has an in-situ concentration that is less than the saturation concentration of the cannabinoid-based incipient,
the cannabinoid-based target component has an in-situ concentration that is less than the saturation concentration of the cannabinoid-based target component,
the in-situ concentration of the cannabinoid-based incipient and the in-situ concentration of the cannabinoid-based target component, taken together, is greater than the saturation concentration of the cannabinoid-based target component, yet the cannabinoid-based vape oil is flowable, and
the cannabinoid-based target component has an in-actio concentration that is greater than the saturation concentration of the cannabinoid-based target component, such that the cannabinoid-based vape oil has a high-effective concentration of the cannabinoid-based target component.

50. The composition of claim 49, wherein the cannabinoid-based incipient is cannabidiolic acid (CBDA) and the cannabinoid-based target component is cannabidiol (CBD).

51. The composition of claim 50, wherein the cannabinoid-based incipient and the cannabinoid-based target component are present in a ratio of between about 1.6:1.0 and about 1.0:1.0, on a weight basis.

52. The composition of claim 50, wherein the cannabinoid-based incipient accounts for between about 20 wt. % and about 70 wt. % of the composition.

53. The composition of claim 50, wherein the cannabinoid-based target component accounts for between about 30 wt. % and about 50 wt. % of the composition.

54. The composition of claim 52, wherein the cannabinoid-based target component accounts for between about 30 wt. % and about 50 wt. % of the composition.

55. The composition of claim 54, wherein the cannabinoid-based incipient accounts for between about 45 wt. % and about 55 wt. % of the composition and the cannabinoid-based target component accounts for between about 30 wt. % and about 40 wt. % of the composition.

56. The composition of claim 55, wherein the cannabinoid-based incipient and/or the cannabinoid-based target component is from a hemp extract.

57. The composition of claim 50, wherein the cannabinoid-based incipient and the cannabinoid-based target component are present in a ratio of between about 1.0:1.3 and about 1.0:2.4, on a weight basis.

58. The composition of claim 57, wherein the cannabinoid-based incipient accounts for between about 26 wt. % and about 40 wt. % of the composition and the cannabinoid-based target component accounts for between about 50 wt. % and about 63 wt. % of the composition.

59. The composition of claim 50, wherein the cannabinoid-based incipient accounts for about 30 wt. % of the composition.

60. The composition of claim 50, wherein the cannabinoid-based target component accounts for about 60 wt. % of the composition.

61. The composition of claim 59, wherein the cannabinoid-based target component accounts for about 60 wt. % of the composition.

62. The composition of claim 61, wherein the cannabinoid-based incipient and/or the cannabinoid-based target component is from a distillate.

63. A cannabinoid composition, comprising:
cannabidiolic acid (CBDA); and
cannabidiol (CBD);
wherein:
the CBDA accounts for between about 20.0 wt. % and about 70.0 wt. % of the composition,
the CBD accounts for between about 30.0 wt. % and about 50.0 wt. % of the composition, and
the composition is flowable within a vape device.

64. The composition of claim 63, wherein the CBDA and the CBD are present in a ratio of between about 1.6:1.0 and about 1.0:1.0, on a weight basis.

65. The composition of claim 63, wherein the CBDA accounts for between about 45 wt. % and about 55 wt. % of the composition.

66. The composition of claim 65, wherein the CBD accounts for between about 30 wt. % and about 40 wt. % of the composition.

67. The composition of claim 66, wherein the CBDA and/or CBD is from a hemp extract.

68. A cannabinoid composition, comprising:
cannabidiolic acid (CBDA); and
cannabidiol (CBD);
wherein:
the CBDA accounts for between about 26 wt. % and about 40 wt. % of the composition,
the CBD accounts for between about 50 wt. % and about 63 wt. % of the composition, and the composition is flowable within a vape device.

69. The composition of claim 68, wherein the CBDA and the CBD are present in a ratio of between about 1.0:1.3 and about 1.0:2.4.

70. The composition of claim 68, wherein the cannabinoid-based incipient accounts for about 30 wt. % of the composition.

71. The composition of claim 68, wherein the cannabinoid-based target component accounts for about 60 wt. % of the composition.

72. The composition of claim 70, wherein the cannabinoid-based target component accounts for about 60 wt. % of the composition.

73. The composition of claim 72, wherein the CBDA and/or CBD is from a distillate.
